(19) United States
(12) Patent Application Publication
(10) Pub. No.: US 2026/0109725 A1
(43) Pub. Date: Apr. 23, 2026

(54) MACROLIDE SENOLYTIC COMPOUNDS

(71) Applicant: LUNELLA BIOTECH, INC., Ottawa, Ontario (CA)

(72) Inventors: Michael P. LISANTI, Didsbury Village, Greater Manchester (GB); Federica SOTGIA, Didsbury Village, Greater Manchester (GB); Béla OZSVARI, Manchester Greater Manchester (GB); Jussi KANGASMETSA, Cambridge Cambridgeshire (GB)

(21) Appl. No.: 18/870,596

(22) PCT Filed: May 30, 2023
(86) PCT No.: PCT/IB2023/055545

§ 371 Date: Nov. 29, 2024

Related U.S. Application Data
(60) Provisional application No. 63/347,826, filed on Jun. 1, 2022.

(51) Int. Cl. C07H 17/08; A61K 31/7052; A61P 35/00; A61P 35/04; A61P 43/00

(52) U.S. Cl. CPC C07H 17/08; A61K 31/7052; A61P 35/00; A61P 35/04; A61P 43/00

(57) ABSTRACT

Disclosed herein are macrolide compounds having senolytic activity. These compounds selectively target senescent cells, and may be used to reduce or eradicate senescent cells in a subject, and/or delay the onset of aging. These compounds also inhibit propagation of cancer stem cells, and may be used to treat cancer, and in particular inhibit the propagation of cancer stem cells responsible for metastasis and tumor recurrence. Formulae (I) & (II):

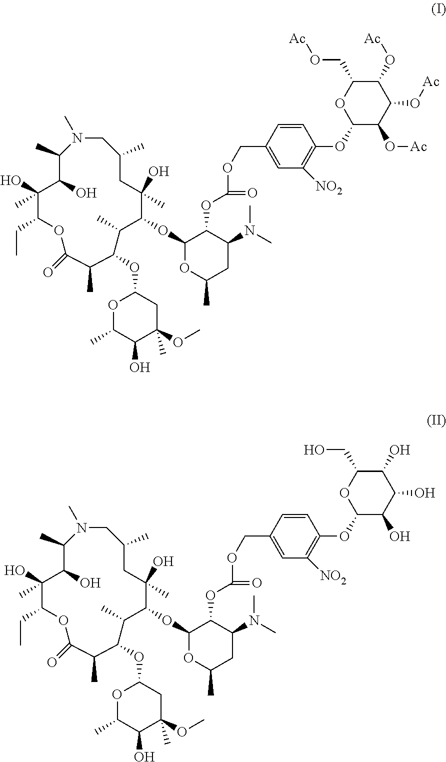

### RELATED APPLICATIONS

N/A

### FIELD

The present disclosure relates to macrolide compounds that selectively eradicate senescent cells and inhibit the propagation of cancer stem cells (CSCs).

### BACKGROUND

The biological process of aging continues to receive significant attention in the scientific and medical research communities. Physiologic aging relates, at least in part, to an increase in the rate of oxidative damage to cellular components, including DNA, lipids, proteins, and the like. The increased oxidative damage creates an imbalance that disrupts self-regulating processes at the cellular level. Further, aging correlates to an accumulation of lipofuscin in neuron cytoplasm. Modern research also indicates that aging is a consequence of naturally occurring DNA damage, resulting in abnormal DNA alterations, accumulating over time. Both mitochondrial and nuclear DNA damage can contribute to aging, indirectly through increasing apoptosis and cellular senescence, and directly by increasing cell dysfunction. Accumulated DNA damage can lead to loss of cells and, in surviving cells, loss of gene expression and mutation-effects that, in infrequently dividing cells, produce indicia of aging. Cellular senescence results when aged cells cease cellular division, believed to occur following various environmental damaging events, abnormal cell growth, autophagy, and oxidative stress, among other factors. Senescence Associated Secretory Phenotype (“SASP”) is a characteristic of senescent cells, and lead to a proteotoxic impairment of healthy cell function, including inflammatory or anti-inflammatory and tumor or anti-tumor effects, depending on a host of factors. The impact of SASP-related chronic inflammation impacts the immune system's normal ability to remove senescent cells, and cells providing an immune function can be conscripted by SASP into senescent cells. Biomarkers of cellular senescence have been found to accumulate as mammals age, and contribute to a wide range of age-related diseases, including Alzheimer's, lateral sclerosis, and type 2 diabetes. And with respect to frequently dividing cells, accumulated DNA damage can become a prominent cause of cancer.

Aging thus increases the likelihood of developing cancer, and researchers have struggled to develop new anti-cancer and anti-aging or senolytic treatments. Conventional cancer therapies (e.g. irradiation, alkylating agents such as cyclophosphamide, and anti-metabolites such as 5-Fluorouracil) have attempted to selectively detect and eradicate fast-growing cancer cells by interfering with cellular mechanisms involved in cell growth and DNA replication. Other cancer therapies have used immunotherapies that selectively bind mutant tumor antigens on fast-growing cancer cells (e.g., monoclonal antibodies). Unfortunately, tumors often recur following these therapies at the same or different site(s), indicating that not all cancer cells have been eradicated. Cancer stem cells, in particular, survive for various reasons, and lead to treatment failure. Relapse may be due to insufficient chemotherapeutic dosage and/or emergence of cancer clones resistant to therapy. Hence, novel cancer treatment strategies are needed that overcome the deficiencies of conventional therapies.

Advances in mutational analysis have allowed in-depth study of the genetic mutations that occur during cancer development. Despite having knowledge of the genomic landscape, modern oncology has had difficulty with identifying primary driver mutations across cancer subtypes. The harsh reality appears to be that each patient's tumor is unique, and a single tumor may contain multiple divergent clone cells. What is needed, then, is a new approach that emphasizes commonalities between different cancer types. Targeting the metabolic differences between tumor and normal cells holds promise as a novel cancer treatment strategy. An analysis of transcriptional profiling data from human breast cancer samples revealed more than 95 elevated mRNA transcripts associated with mitochondrial biogenesis and/or mitochondrial translation. Sotgia et al., Cell Cycle, 11 (23): 4390-4401 (2012). Additionally, more than 35 of the 95 upregulated mRNAs encode mitochondrial ribosomal proteins (MRPs). Proteomic analysis of human breast cancer stem cells likewise revealed the significant overexpression of several mitoribosomal proteins as well as other proteins associated with mitochondrial biogenesis. Lamb et al., Oncotarget, 5 (22): 11029-11037 (2014).

Cancer cell mitochondrial metabolism has been the target of recent explorative research, with respect to both searching for anti-cancer therapeutic targets and senolytic therapeutic targets. Mitochondria are extremely dynamic organelles in constant division, elongation and connection to each other to form tubular networks or fragmented granules in order to satisfy the requirements of the cell and adapt to the cellular microenvironment. The balance of mitochondrial fusion and fission dictates the morphology, abundance, function and spatial distribution of mitochondria, therefore influencing a plethora of mitochondrial-dependent vital biological processes such as adenosine triphosphate (ATP) production, mitophagy, apoptosis, and calcium homeostasis. In turn, mitochondrial dynamics can be regulated by mitochondrial metabolism, respiration and oxidative stress.

ATP is the universal bioenergetic “currency” of all living cells and tissues, including microorganisms, such as prokaryotic bacteria and eukaryotic yeast. In eukaryotes, mitochondrial organelles function as the “powerhouse” of the cell. Mitochondria generate the vast amount of ATP via the TCA cycle and oxidative phosphorylation (OXPHOS), while glycolysis contributes a minor amount of ATP. Conversely, mitochondrial dysfunction induces ATP-depletion, resulting in mitochondrial-driven apoptosis (programmed cell death) and/or necrosis. Thus, we have proposed that ATP-depletion therapy may be a viable strategy for targeting and eradicating even the “fittest” cancer cells.

In MCF-7 breast cancer cells, mitochondrial-driven OXPHOS contributes to 80-90% of ATP production, while glycolysis only contributes the remaining 10-20%, under normoxic conditions. Therefore, like normal cells, cancer cells are highly dependent on mitochondrial ATP production. However, it still remains largely unknown if ATP levels in cancer cells contribute to undergo 3D anchorage-independent growth and cell migration, two characteristic features of metastatic spread.

What is needed, then, are novel anti-aging compositions and methods that treat aging at the cellular level, overcoming accumulated oxidative and DNA damage and the numerous undesired effects of aging.

Further, what is needed are therapeutic agents that target unhealthy senescent cells and SASP, reducing the accumulation of cellular senescence and offsetting chronic senescence.

What is further needed are therapeutic agents that target a broad range of CSCs, through a feature common to CSCs regardless of cancer type.

What is further needed are therapeutic agents that inhibit the propagation of CSCs, including the circulating tumor cells and tumor-initiating cells that have the potential to cause tumor recurrence and/or metastasis.

### SUMMARY

In view of the foregoing background, it is an object of this disclosure to describe therapeutic agents, or compounds, that may be used to reduce the accumulation of cellular senescence, and inhibit CSC propagation. It is an object of this disclosure to describe therapeutic agents for use in eradicating CSCs and senescent cells. It is an object of this disclosure to describe therapeutic agents for use in preventing and reducing the likelihood of tumor recurrence and metastasis. It is further an object of this disclosure to describe compositions, such as pharmaceutical compositions, and methods for treating and preventing cancer, including tumor recurrence and/or metastasis. It is also an object of this disclosure to describe compositions, such as pharmaceutical compositions, and methods for senolytic therapeutic agents.

Described herein are compounds that may be used as therapeutic agents having anti-cancer activity, pharmaceutical compositions containing such therapeutic agents, methods for synthesizing such compounds, and methods for treating cancer.

The present approach may also be used to treat and/or prevent tumor recurrence and/or metastasis. Anti-cancer treatments often fail because the tumor recurs or metastasizes, particularly after surgery. CSC mitochondrial activity is understood to be, at least in part, responsible for these causes of treatment failure. Embodiments of the present approach may be used in situations where conventional cancer therapies fail, and/or in conjunction with or prior to anti-cancer treatments, to prevent or reduce the likelihood of treatment failure due to tumor recurrence and/or metastasis.

### BRIEF DESCRIPTION OF THE DRAWINGS

FIG. 1 compares SRB assay results for different concentrations of Azithromycin and Compound [I] of the present approach, also referred to as AZM-Gal, on both control MRC5 cells and MRC5 cells treated with BrdU.

FIG. 2 shows xCELLigence data comparing the effect of Compound [I] of the present approach on control MRC5 cells and MRC5 cells treated with BrdU.

FIGS. 3A and 3B show images of MRC-5 fibroblasts without and with BrdU pre-treatment, respectively, treated with Compound [I] at a concentration of 50 μM.

FIG. 4A shows mammosphere formation assay results for MCF-7 cells treated with Compound [I], and FIG. 4B shows mammosphere formation assay results for MCF-7 cells treated with Azithromycin.

FIG. 5 compares SRB assay results for Compound [III] on both control MRC5 cells and MRC5 cells treated with BrdU.

FIG. 6 compares SRB assay results for Azithromycin and Compound [IV] on both control MRC5 cells and MRC5 cells treated with BrdU.

FIG. 7 compares SRB assay results for Compound [V] and Compound [VI] on both control MRC5 cells and MRC5 cells treated with BrdU.

### DESCRIPTION

The following description illustrates embodiments of the present approach in sufficient detail to enable practice of the present approach. Although the present approach is described with reference to these specific embodiments, it should be appreciated that the present approach can be embodied in different forms, and this description should not be construed as limiting any appended claims to the specific embodiments set forth herein. Rather, these embodiments are provided so that this disclosure will be thorough and complete, and will fully convey the scope of the present approach to those skilled in the art.

This description uses various terms that should be understood by those of an ordinary level of skill in the art. The following clarifications are made for the avoidance of doubt.

The term “cancer” refers to physiological conditions in mammals that are typically characterized by uncontrolled cell growth. This definition includes benign and malignant cancers. Examples of cancers include cancer types, lymphomas, blastomas (including medullablastomas and retinoblastomas), sarcomas (including liposarcomas and synovial sarcomas), neuroendocrine tumors (carcinoid tumors, gastrin production Includes, but is not limited to, tumors and islet cell carcinomas), sarcomas, Schwannomas (including acoustic neuroma), medullary carcinomas, adenocarcinomas, melanomas, and leukemia or lymphocyte tumors. Specific examples of cancers include bladder cancer, squamous cell carcinoma, small cell lung cancer, non-small cell lung cancer, lung adenocarcinoma, and lung cancer including squamous epithelial cancer of the lung, peritoneal cancer, hepatocellular carcinoma, gastric cancer including gastrointestinal cancer or stomach cancer, pancreatic cancer, glioblastoma, cervical cancer, ovarian cancer, liver cancer, bladder cancer, liver cancer, breast cancer (including metastatic breast cancer), colon cancer, rectal cancer, colon rectal cancer, endometrial cancer or uterine cancer, salivary adenocarcinoma, kidney cancer (kidney cancer) or kidney cancer (renal cancer), prostatic cancer, genital cancer, thyroid cancer, liver cancer, anal cancer, penis cancer, testicular cancer, esophageal cancer, bile duct tumor, and head and neck cancer and multiple myeloma.

As used herein, the term “tumor” refers to the growth and proliferation of neoplastic cells, whether malignant or benign, including pre-cancerous and cancerous cells and tissues.

The term “metastasis” refers to the spread of cancer from its primary site to other parts of the body. Cancer cells can escape from the primary tumor, penetrate lymph vessels and blood vessels, circulate through the bloodstream, and grow or “metastasize” in distant lesions in normal tissue elsewhere in the body. Metastases can be local or distant. Metastasis is a sequential process that requires tumor cells to escape from the primary tumor, travel through the bloodstream, and stop at distant sites. At this new site, cells can establish a blood supply and grow to form a life-threatening mass. Both irritating and inhibitory molecular pathways within tumor cells control this behavior, and the interaction between tumor cells and host cells at distant sites is also important.

The terms “treat,” “treated,” “treating,” and “treatment” include the diminishment or alleviation of at least one symptom associated or caused by the state, disorder or disease being treated, in particular, cancer. In certain embodiments, the treatment comprises diminishing and/or alleviating at least one symptom associated with or caused by the cancer being treated, by the compound of the invention. In some embodiments, the treatment comprises causing the death of a category of cells, such as senescent cells, SASP cells, or CSCs likely to be involved in metastasis or recurrence, of a particular cancer in a host, and may be accomplished through preventing senescent cells and/or cancer cells from further propagation, and/or inhibiting CSC function through, for example, depriving such cells of mechanisms for generating energy. For example, treatment can be diminishment of one or several symptoms of a cancer, or complete eradication of a cancer. As another example, the present approach may be used to inhibit mitochondrial metabolism in the cancer, eradicate (e.g., killing at a rate higher than a rate of propagation) CSCs in the cancer, eradicate TICs in the cancer, eradicate circulating tumor cells in the cancer, inhibit propagation of the cancer, target and inhibit CSCs, target and inhibit TICs, target and inhibit circulating tumor cells, prevent or reduce the likelihood of, metastasis, prevent recurrence, sensitize the cancer to a chemotherapeutic, sensitize the cancer to radiotherapy, sensitize the cancer to phototherapy. As another example, the treatment can reduce the accumulated senescent cells, and/or reduce the rate of senescent cell accumulation.

In the context of tumor recurrence and/or metastasis, the term “prevent” and “reduce the likelihood of” refer to reducing, in a subject, the presence of CSCs, TICs, and circulating tumor cells, likely to be involved in recurrence or metastasis, to a level at which tumor recurrence and/or metastasis from the primary site is unlikely, relative to a control (i.e., no treatment to prevent or reduce the likelihood of tumor recurrence and/or metastasis). In practice, a treatment to prevent and/or reduce the likelihood of tumor recurrence and/or metastasis as described herein targets and inhibits or eradicates CSCs, TICs, inhibit circulating tumor cells.

The terms “cancer stem cell” and “CSC” refer to the subpopulation of cancer cells within tumors that have capabilities of self-renewal, differentiation, and tumorigenicity when transplanted into an animal host. Compared to “bulk” cancer cells, CSCs have increased mitochondrial mass, enhanced mitochondrial biogenesis, and higher activation of mitochondrial protein translation. As used herein, a “circulating tumor cell” is a cancer cell that has shed into the vasculature or lymphatics from a primary tumor and is carried around the body in the blood circulation. The CellSearch Circulating Tumor Cell Test may be used to detect circulating tumor cells.

The phrase “pharmaceutically effective amount,” as used herein, indicates an amount necessary to administer to a host, or to a cell, tissue, or organ of a host, to achieve a therapeutic result, such as regulating, modulating, or inhibiting protein kinase activity, e.g., inhibition of the activity of a protein kinase, or treatment of cancer. A physician or veterinarian having ordinary skill in the art can readily determine and prescribe the effective amount of the pharmaceutical composition required for a given subject, using methods well-known and available in the art. For example, the physician or veterinarian could start doses of the compounds of the invention employed in the pharmaceutical composition at levels lower than that required in order to achieve the desired therapeutic effect and gradually increase the dosage until the desired effect is achieved. The determination of a pharmaceutically effective amount is deemed to be within the purview of the person having an ordinary level of skill in the art, having reviewed this disclosure.

As used herein, the phrase “therapeutic agent” refers to an embodiment of the compound described herein, which may include a pharmaceutically acceptable salt or isotopic analog thereof. It should be appreciated that the therapeutic agent may be administered to the subject through any suitable approach, as would be known to those having an ordinary level of skill in the art. It should also be appreciated that the amount of therapeutic agent and the timing of its administration may be dependent on the individual subject being treated (e.g., the age and body mass, among other factors), on the manner of administration, on the pharmacokinetic properties of the particular therapeutic agent, and on the judgment of the prescribing physician. Thus, because of subject to subject variability, any dosages described herein are intended to be initial guidelines, and the physician can titrate doses of the therapeutic agent to achieve the treatment that the physician considers appropriate for the subject. In considering the degree of treatment desired, the physician can balance a variety of factors such as age and weight of the subject, presence of preexisting disease, as well as presence of other diseases. Pharmaceutical formulations can be prepared for any desired route of administration including, but not limited to, oral, intravenous, or aerosol administration, as discussed in greater detail below.

The phrase “pharmaceutically acceptable carrier” as used herein, means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose: (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrase “pharmaceutically acceptable salt” to the relatively non-toxic, inorganic and organic base addition salts of the compounds of the present approach. A pharmaceutically acceptable salt may be formed by, for example, reacting a compound in its free acid form with a base, such as hydroxide or carbonate of a pharmaceutically-acceptable metal cation, with ammonia or with a pharmaceutically-acceptable amine. Representative alkali or alkaline earth salts include sodium, potassium, calcium, magnesium, and aluminum salts, for example. Examples of amines that may be used for base addition salts include ethylamine, diethylamine, ethylenediamine, ethanolamine, diethanolamine, piperazine. It should be appreciated that other salts may be used, of course, and that the person of ordinary skill in the art may use methods known in the art for identifying suitable salt forms, without departing from the present approach.

Senescence is a clear hallmark of normal chronological aging. Senescence involves potentially irreversible cell cycle arrest, via the induction of CDK-inhibitors, such as p16-INK4A, p19-ARF, p21-WAF and p27-KIP1, as well as the onset of SASP (senescence-associated secretory phenotype), and the induction of key lysosomal enzymes (Beta-Galactosidase) and Lipofuscin, an established aging-pigment. Interestingly, SASP results in the secretion of a wide array of inflammatory cytokines, such as IL-1-beta and IL-6, allowing senescent cells to “contagiously” spread the senescence phenotype from one cell type to another, systemically throughout the body, via chronic inflammation. Such chronic inflammation can also promote the onset of cancer, as well as drive tumor recurrence and metastasis.

Described herein are compounds having potency against, and high selectivity for, senescent cells. Compounds of the present approach may be used as senolytics, e.g., therapeutic agents to eradicate senescent cells. Further, compounds of the present approach show significantly less antibiotic activity when compared to reference compounds. Accordingly compounds of the present approach do not contribute to potential antibiotic resistance.

Some embodiments of the present approach take the form of a compound having the structure of Compound [I] shown below, in which ‘Ac’ denotes an acetyl group.

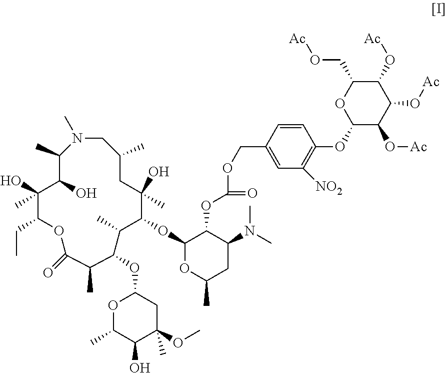 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)CN(C)[C@H](C)[C@@H](O)[C@]1(C)O]

Compound [I], also referred to as AZM-Gal, has the IUPAC name [(2R,3S,4S,5R,6S)-3,4,5-triacetoxy-6-[4-[[(2S,3R,4S,6R)-4-(dimethylamino)-2-[[(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxycarbonyloxymethyl]-2-nitro-phenoxy]tetrahydropyran-2-yl]methyl acetate. The galactose is joined via the hydroxide of the desosamine ring.

The structure shown below, Compound [II] (also referred to as de-acetylated AZM-Gal) has comparable activity to Compound [I].

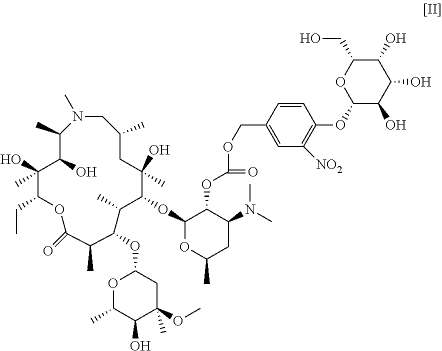 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](CO)[C@H](O)[C@H](O)[C@H]3O)c([N+](=O)[O-])c2)[C@](C)(O)C[C@@H](C)CN(C)[C@H](C)[C@@H](O)[C@]1(C)O]

The compounds disclosed herein have exhibited senolytic activity via clinical assays. For example, in one assay bromodeoxyuridine (5-bromo-2′-deoxyuridine), also known as BrdU, was used to induce senescence in a cell population. BrdU is an analog of the nucleoside thymidine commonly used to identify proliferating cells. BrdU induces controlled DNA damage, and drives cells towards senescence with high efficiency. In the BrdU assay, normal fibroblasts are subjected to prolonged culture (8-days) in the presence of BrdU at 100 μM to induce controlled DNA-damage and senescence. In demonstrative embodiments, the inventors used two independent normal, non-immortalized, human fibroblast cell lines, MRC-5 lung cells, in the BrdU-based assay. Senolytic activity was assessed using the sulforhodamine B assay, also known in the art as the SRB assay. This assay measures the amount of protein remaining attached to the tissue-culture dishes, and is a surrogate marker for cell viability.

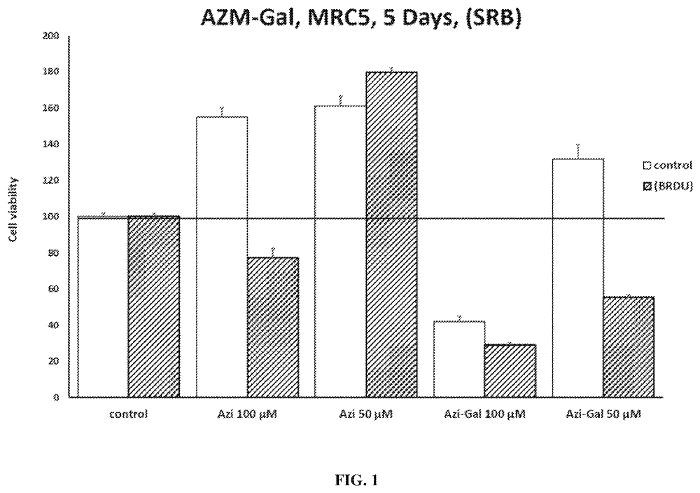

FIG. 1 compares SRB assay results for different concentrations of Azithromycin (100 UM and 50 μM) and Compound [I] (100 μM and 50 μM) of the present approach, on both control MRC-5 cells and MRC-5 cells treated with BrdU. For these data, MRC-5 cells were pre-treated with BrdU for 8 days (to induce senescence), before they were exposed to Azithromycin or Compound [I] (labelled as “Azi-Gal” in FIG. 1) for another 5 days. After that, the SRB assay was performed to determine the effects of the drug on cell viability, using normal MRC-5 cells as the control. As can be seen, Azithromycin at 100 μM had no effect on the viability of normal MRC-5 lung fibroblasts, but selectively killed senescent MRC-5 fibroblasts. However, at 50 μM Azithromycin had no effect on either the control cells or senescent cells. In contrast, Compound [I] showed exceptional selectivity towards senescent MRC-5 fibroblasts at 50 μM, and was effective against both the control cells and the senescent cells at 10 μM.

Senescent cells undergo the so-called senescence-associated secretory phenotype (SASP), which involves dramatic increases in the synthesis and secretion of proteins. The xCELLigence assay system was used to assess whether protein measurement assays under-estimate the senolytic activity of tested compounds. The xCELLigence assay system does not depend on proteins, and instead uses electrical impedance to continuously measure cell proliferation in real time. The real-time xCELLigence assay system thus compliments the more static SRB assay, and offers a more direct visualization of the potential senolytic effects of compounds during drug screening.

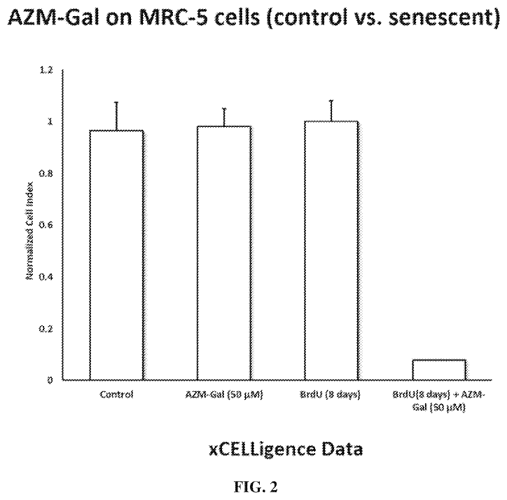

The xCELLigence assay was used to confirm senolytic activity for Compound [I]. MRC-5 fibroblasts were used for the assay. FIG. 2 shows xCELLigence data comparing the effect of Compound [I] of the present approach on control MRC-5 cells and MRC-5 cells treated with BrdU. The data is expressed as the final cell index, the average±the standard error of mean, for the control cells, control cells treated with Compound [I], BrdU-treated control cells, and BrdU-treated fibroblasts subjected to Compound [I] at 50 μM, respectively. The senescent MRC-5 cells were pretreated with BrdU for 8 days to induce senescence, before exposure to Compound [I] for another 5 days. Compared to the control, Compound [I] treatment had no effect on the viability of normal fibroblasts, but killed over 90% of the BrdU-treated fibroblasts. These data confirm that Compound [I] has exceptional senolytic activity.

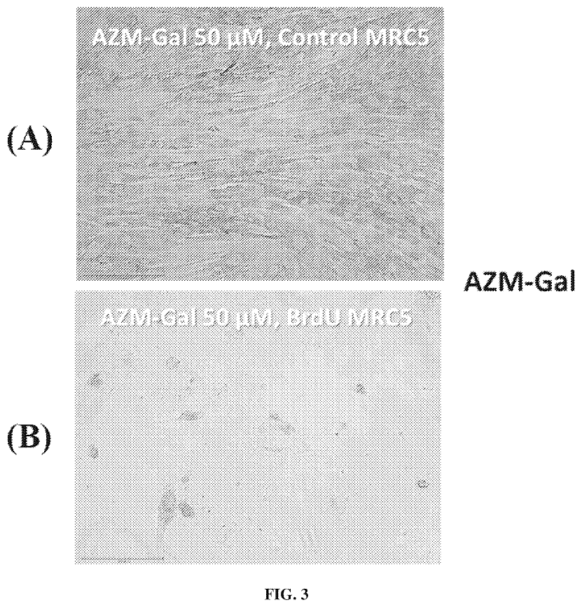

FIGS. 3A and 3B are images of MRC-5 fibroblasts without and with BrdU pre-treatment, respectively, treated with Compound [I] at a concentration of 50 μM. These images show that Compound [I] had little effect on the normal MRC-5 cells, but induced cell death in senescent MRC-5 cells. The scale bar in the upper-right of FIGS. 3B and 3C represents 20 μm.

These results show that the compounds disclosed herein have senolytic activity and may be used as senolytic agents in a pharmaceutical composition. For example, the compound may be used to eradicate senescent cells in a subject. Some embodiments of the present approach may take the form of a method for delaying the onset of an age-related disease in a subject. The age-related disease may be at least one of atherosclerosis, arthritis, cancer, cardiovascular disease, cataract, dementia, diabetes, hair loss, hypertension, inflammatory disease, kidney disease, muscular atrophy, neurological disease, osteoarthritis, osteoporosis, pulmonary disease, vertebral disc degeneration, and alopecia. For example, the age-related disease may be a neurological disease, such as mild cognitive impairment, motor neuron dysfunction, Alzheimer's disease, Parkinson's disease, and macular degeneration. In such embodiments, a therapeutic amount of a senolytic agent, as described, herein may be administered to the subject. In some embodiments, the senolytic agent may be administered with another therapeutic agent, as described herein. The senolytic agent may be administered at the onset, i.e., at or shortly after the diagnosis of an age-related disease. Alternatively, the senolytic agent may be administered routinely after diagnosis, and the frequency and dosage may be determined using techniques known in the art. In some embodiments, the senolytic agent may be administered prior to onset, particularly where an age-related disease is expected or likely to occur in a subject (e.g., due to genetic markers or other biological markers).

The compounds disclosed herein may also be used as therapeutic agent to selectively eradicate CSCs for treating and/or preventing tumor recurrence and/or metastasis. The data demonstrates that the compounds disclosed herein have anti-cancer activity, and are suitable for use as therapeutic agents for anti-cancer treatments, including treating and/or preventing tumor recurrence and metastasis. Data described herein demonstrates the anti-cancer activity through inhibition of MCF-7 cells via the mammosphere formation assay. This assay measures the amount of residual protein that adheres to tissue culture dishes and is a surrogate marker of cell viability.

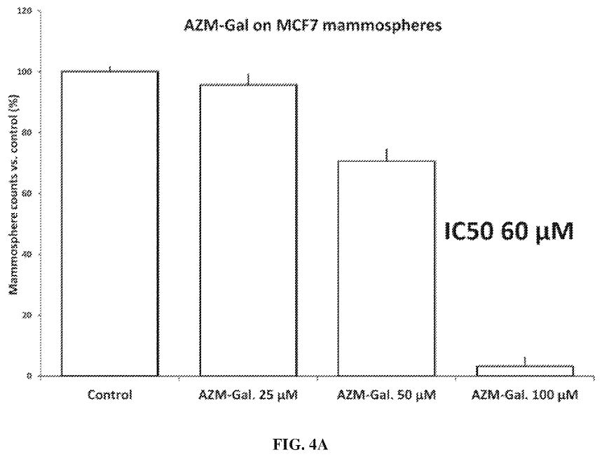

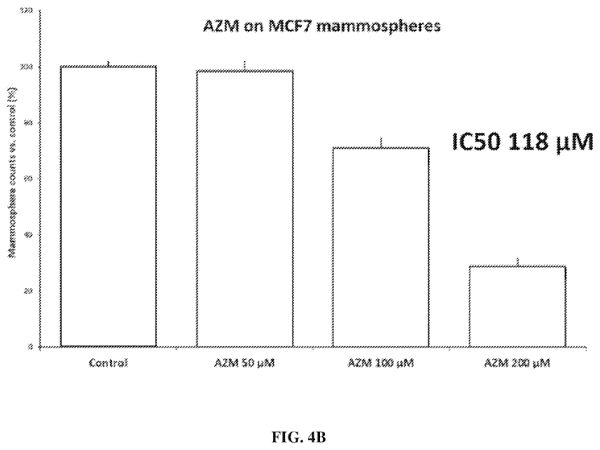

FIG. 4A shows mammosphere formation assay results for MCF-7 cells treated with Compound [I], and FIG. 4B shows mammosphere formation assay results for MCF-7 cells treated with Azithromycin. The data show that Compound [I] had an IC₅₀ of 60 μM, and nearly complete inhibition at a concentration of 100 μM. Azithromycin, on the other hand, had an IC₅₀ of 118 μM, and even at a concentration of 200 μM, inhibited only about 30% of the MCF-7 cells compared to the control. These results demonstrate the superior potency of Compound [I], at inhibiting MCF-7 propagation.

However, modification of known senolytic compounds with a galactose moiety does not always result in improved senolytic activity. Compound [III], also referred to as AZM-Succinate or AZM-Succ, was synthesized as described herein. As can be seen, in Compound [III] the galactose is joined at the hydroxide of the desosamine, via succinate. However, as discussed below, Compound [III] showed significantly less senolytic activity compared to Azithromycin and Compound [I].

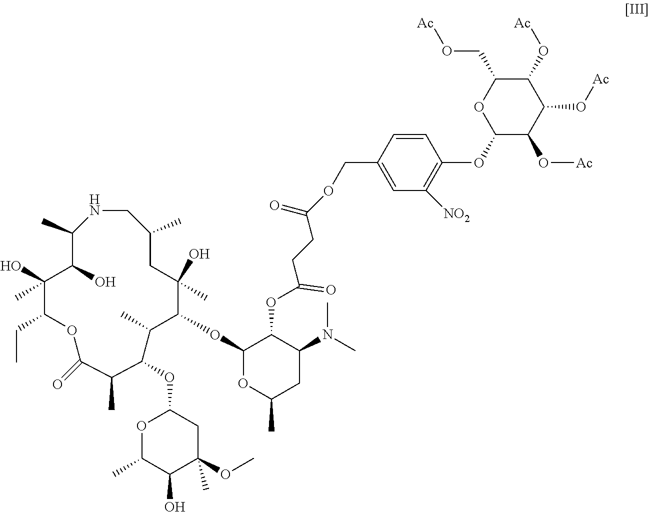 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)CCC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)CN[C@H](C)[C@@H](O)[C@]1(C)O]

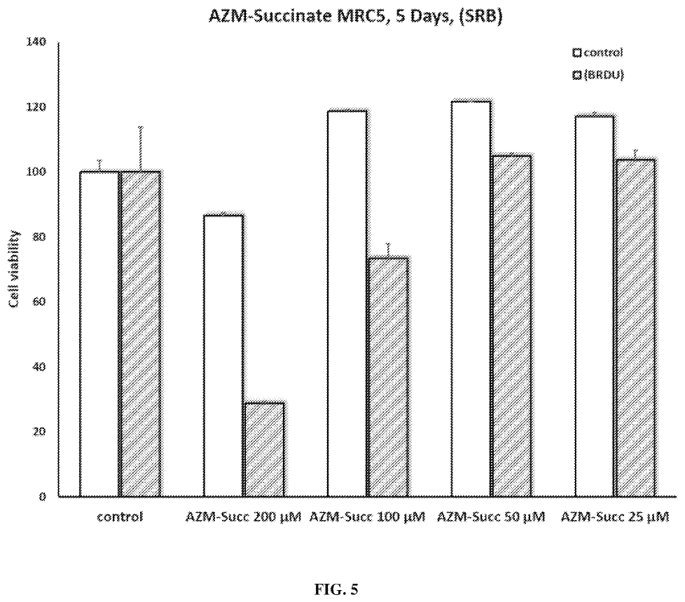

FIG. 5 shows compares SRB assay results for Compound [III] on both control MRC5 cells and MRC5 cells treated with BrdU. The addition of the galactose moiety reduced the potency of the parent compound, Azithromycin, by about half. This demonstrates that merely conjugating the galactose moiety does not improve the senolytic activity of the base compound.

Additionally, modification with a galactose moiety does not always improve a parent compound's selectivity for senescent cells. Compound [IV], shown below and referred to as Des-AZM-Gal, is demonstrative. As can be seen, the galactose is joined via the amine of the desosamine ring of the macrolide structure.

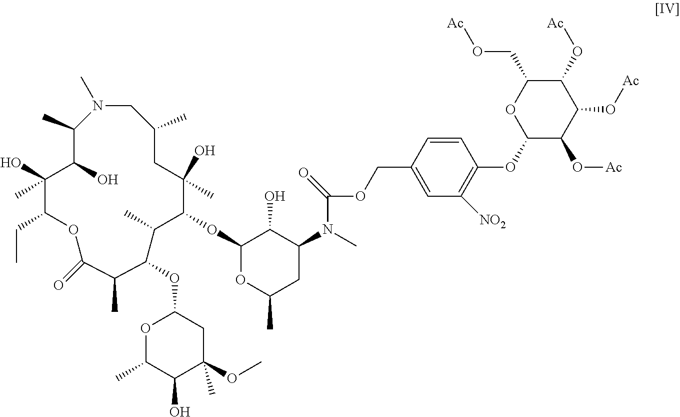 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C(=O)OCc3ccc(O[C@@H]4O[C@H](COC(C)=O)[C@H](OC(C)=O)[C@H](OC(C)=O)[C@H]4OC(C)=O)c([N+](=O)[O-])c3)[C@H]2O)[C@](C)(O)C[C@@H](C)CN(C)[C@H](C)[C@@H](O)[C@]1(C)O]

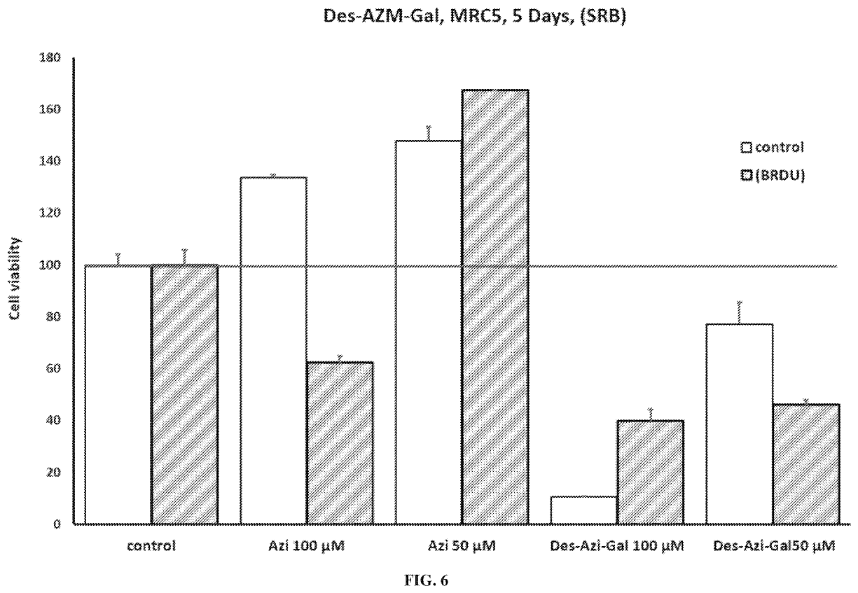

FIG. 6 compares SRB assay results for Azithromycin and Compound [IV] on both control MRC5 cells and MRC5 cells treated with BrdU. As can be seen, Compound [IV] showed no little selectivity for senescent cells over normal, non-senescent cells.

Conjugation with galactose causes some compounds to not only lose senolytic and antibiotic activity, but also target and kill normal, non-senescent cells. Compound [V], shown below and also referred to as Roxy-Gal, is a conjugate of Roxithromycin and Galactose.

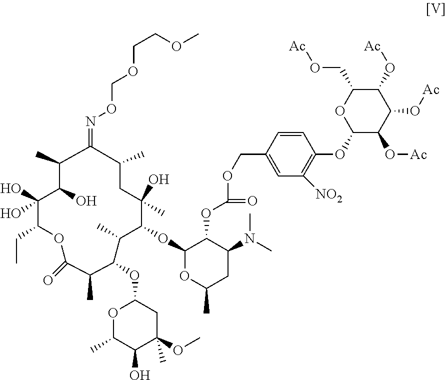

Compound [VI], shown below and referred to as Erythro-Gal, is a conjugate of Erythromycin and galactose. Compounds [V] and [VI] are conjugated using the same general approach as Compound [I], via the hydroxide of the desosamine ring of the macrolide structure. The resulting activity, however, is considerably different, and illustrates the unique properties of Compounds [I] and [II].

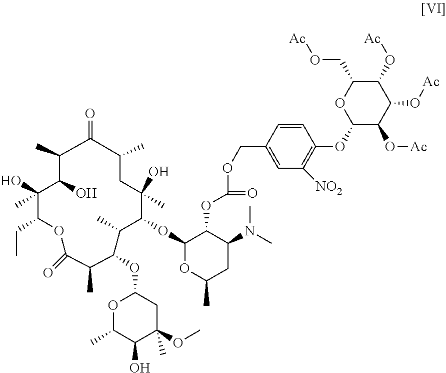 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)C(=O)[C@H](C)[C@@H](O)[C@]1(C)O]

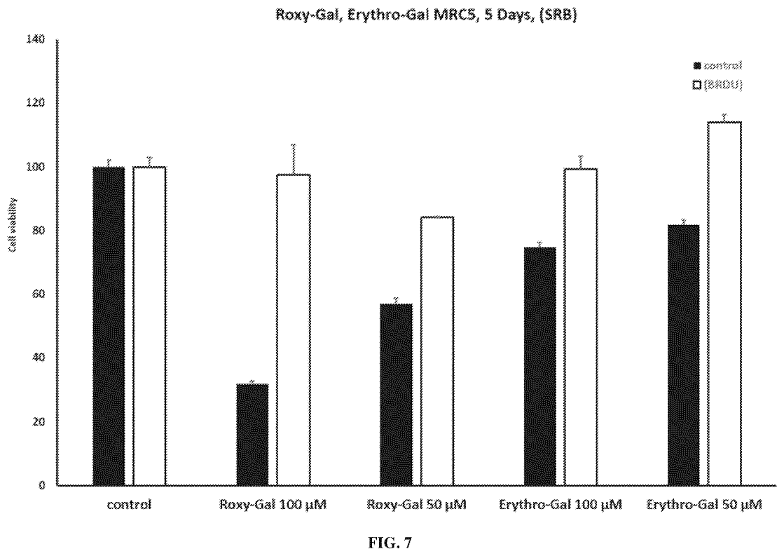

FIG. 7 compares SRB assay results for Compound [V] and Compound [VI] on both control MRC5 cells and MRC5 cells treated with BrdU. Compared to Roxithromycin and Erythromycin, these compounds show a preference for the normal, non-senescent control cells, and had little effect on the senescent cells. These data contrast sharply with the activity demonstrated with Compounds [I] and [II].

Compounds of the present approach, Compound [I] and Compound [II], have less antibiotic activity than existing macrolide antibiotics, such as azithromycin and erythromycin. This is advantageous because use of the compounds of the present approach as therapeutic agents will have less of an impact on the development of antibiotic resistance. For example, embodiments of the present approach were screened for antibiotic activity, using the in vitro broth microdilution assay. In this assay, the Minimum Inhibitory Concentration (MIC) is defined as the lowest concentration of an agent that completely inhibits visible growth in vitro of the microorganism. The assay conditions, described by the Clinical and Laboratory Standards Institute were used for preparation of the inoculum, growth medium, and end point reading. Test substance was dissolved in 100% DMSO, suspended completely by vortexing, diluted by 2-fold serial titrations in the same vehicle, for a total of 11 test concentrations. A 4 μL aliquot of each dilution was added to 196 μL of broth medium seeded with the organism suspension in wells of a 96 well plate (bacterial count: 2-8×10(5) colony forming units/mL final). The final volume was 200 μL in each well and the final DMSO concentration was 2 percent. Test concentrations were 0.1 to 100 μM. Following incubation, the test plates were visually examined and wells were scored for growth or complete growth inhibition to define the minimum inhibitory concentration. Each test substance was evaluated with replicates. Vehicle controls and an active reference agent were used as blank and positive controls. Results are shown in Table 1. Note that MRSA represents methicillin-resistant Staphylococcus aureus, and VRE represents vancomycin resistant Enterococcus.

TABLE 1
Antimicrobial potency results.

| | | | | MIC, μM | |
|---|---|---|---|---|---|
| | | | Strain | | Compound |
| No. | Species | Strain ID | information | AZM | [I] |
| 1 | Enterococcus faecalis | ATCC 29212 | QC strain | 6.25 | 25 |
| 2 | Escherichia coli | ATCC 25922 | QC strain | 6.25 | 12.5 |
| 3 | Klebsiella pneumoniae | ATCC 43816 | — | 25 | 50 |
| 4 | Pseudomonas aeruginosa | ATCC 27853 | QC strain | 100 | >100 |
| 5 | Staphylococcus aureus | ATCC 29213 | QC strain | 1.56 | 6.25 |
| 6 | Staphylococcus aureus | ATCC 33591 | MRSA | >100 | >100 |
| 7 | Acinetobacter baumannii | ATCC 17978 | — | 6.25 | 100 |
| 8 | Streptococcus | ATCC 49619 | QC strain | 0.39 | 0.78 |
| | pneumoniae | | | | |
| 9 | Haemophilus influenzae | ATCC 49247 | QC strain | 3.125 | 3.125 |
| 10 | Enterococcus faecium | ATCC | VanA VRE | >100 | >100 |
| | | 700221 | | | |
| 11 | Escherichia coli | JW5503 | DEL-tolC | 1.56 | 6.25 |

Table 1 shows that the antimicrobial potency (expressed as MIC) of the tested embodiment were higher than the control, azithromycin. The MIC for Compound [I] AZM-Gal is considerably higher for nearly every species tested. This is demonstrative for embodiments of the present approach—the compounds disclosed herein have less antibiotic activity compared to macrolide antibiotics.

The following paragraphs provide example synthesis schemes for embodiments of the present approach. Synthesis products were confirmed using liquid chromatography and mass spectroscopy (LC-MS). The LC column was a Waters Sunfire C18 30×4.6 mm, using a gradient eluent of 20-100% acetonitrile/water containing 0.05% formic acid. Time: 0-10 min. The following abbreviation are used in the synthesis examples: Acetonitrile (MeCN), methanol (MeOH), dichloromethane (DCM), dichloroethane (DCE), isopropanol (IPA), sodium borohydride (NaBH₄), sodium carbonate Na₂CO₃, ammonium chloride (NH₄Cl), 4-dimethylaminopyridine (DMAP), N-(3-Dimethylaminopropyl)-N′-ethylcarbodiimide hydrochloride (EDC HCl), triethylamine (TEA).

### Example 1—Intermediary [(2R,3R,4S,5R,6S)-3,4,5-triacetoxy-6-(4-formyl-2-nitro-phenoxy)tetrahydropyran-2-yl]methyl acetate

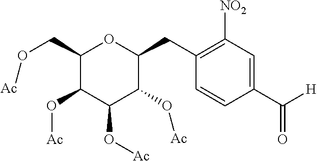 [SMILES: CC(=O)OC[C@H]1O[C@@H](Cc2ccc(C=O)cc2[N+](=O)[O-])[C@H](OC(C)=O)[C@@H](OC(C)=O)[C@H]1OC(C)=O]

To a stirred suspension of Ag₂O (8.0 g, 34.52 mmol) and tetra-O-acetyl-a-D-glucopyranosyl bromide (1.26 g, 7.55 mmol) in MeCN (50 ml) a solution of 4-hydroxy-3-nitrobenzaldehyde (3.10 g, 7.55 mmol) in acetonitrile (35 ml) was added at room temperature. The mixture was stirred at room temperature for 4 hours, solid residue was removed by filtration and the solvent was evaporated under reduced pressure to yield a crude product. Purification on silica gel (2-4% MeOH in DCM) afforded [(2R,3R,4S,5R,6S)-3,4,5-triacetoxy-6-(4-formyl-2-nitro-phenoxy) tetrahydropyran-2-yl]methyl acetate (3.61 g). LC-MS 515.2 [M+H₂O]⁺, RT 4.25 min.

### Example 2—Intermediary [(2R,3R,4S,5R,6S)-3,4,5-triacetoxy-6-[4-(hydroxymethyl)-2-nitro-phenoxy]tetrahydropyran-2-yl]methyl acetate

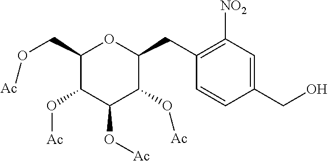 [SMILES: CC(=O)OC[C@H]1O[C@@H](Cc2ccc(CO)cc2[N+](=O)[O-])[C@H](OC(C)=O)[C@@H](OC(C)=O)[C@@H]1OC(C)=O]

To a stirred ice-cold solution of [(2R,3R,4S,5R,6S)-3,4,5-triacetoxy-6-(4-formyl-2-nitro-phenoxy)tetrahydropyran-2-yl]methyl acetate (3.00 g, 6.03 mmol) in a mixture of dry DCM (30 ml) and dry IPA (8.5 ml) under nitrogen atmosphere NaBH₄ (0.50 g, 13.22 mmol) was added and the mixture was stirred for 3.5 hours. A solution of saturated NH₄Cl (70 ml) was added to the stirred mixture, stirring was continued for 5 minutes, the product was extracted with twice with DCM, the combined extracts was washed with brine, dried over Na₂CO₃, the solid residue was removed by filtration and the solvent was evaporated under reduced pressure to [(2R,3R,4S,5R,6S)-3,4,5-triacetoxy-6-[4-(hydroxymethyl)-2-nitro-phenoxy]tetrahydropyran-2-yl]methyl acetate (2.60 g). LC-MS 517.2 [M+H₂O]⁺, RT 3.91 min.

### Example 3—Intermediary [(2R,3R,4S,5R,6S)-3,4,5-triacetoxy-6-[4-(chlorocarbonyloxymethyl)-2-nitro-phenoxy]tetrahydropyran-2-yl]methyl acetate

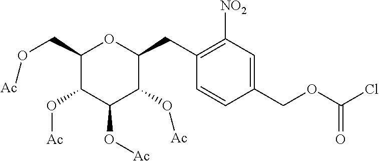 [SMILES: CC(=O)OC[C@H]1O[C@@H](Cc2ccc(COC(=O)Cl)cc2[N+](=O)[O-])[C@H](OC(C)=O)[C@@H](OC(C)=O)[C@@H]1OC(C)=O]

To a stirred ice cold suspension of [(2R,3R,4S,5R,6S)-3,4,5-triacetoxy-6-[4-(hydroxymethyl)-2-nitro-phenoxy]tetrahydropyran-2-yl]methyl acetate (0.25 g, 0.50 mmol) and K₂CO₃ (0.44 g, 3.00 mmol) in dry MeCN (5 ml) under nitrogen atmosphere an excess of phosgene (1.60 ml, 3.00 mmol) was added and the mixture was stirred at +5° C. for 2 hours. The solid residue was removed by filtration through Celite and the solvent was evaporated under reduced pressure to yield a crude product [(2R,3R,4S,5R,6S)-3,4,5-triacetoxy-6-[4-(chlorocarbonyloxymethyl)-2-nitro-phenoxy]tetrahydropyran-2-yl]methyl acetate.

### Example 4—Intermediary [(2R,3S,4S,5R,6S)-3,4,5-triacetoxy-6-(4-formyl-2-nitro-phenoxy)tetrahydropyran-2-yl]methyl acetate

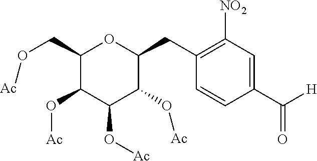 [SMILES: CC(=O)OC[C@H]1O[C@@H](Cc2ccc(C=O)cc2[N+](=O)[O-])[C@H](OC(C)=O)[C@@H](OC(C)=O)[C@H]1OC(C)=O]

The title compound was prepared according to Ghosh Ajit, K., at al. A daunorubicin b-galactoside prodrug for use in conjunction with gene-directed enzyme prodrug therapy Tetrahedron Lett. 2000, 41, 4871-4874. LC-MS 515.2 [M+H₂O]⁺, RT 4.16 min.

### Example 5—Intermediary [(2R,3S,4S,5R,6S)-3,4,5-triacetoxy-6-[4-(hydroxymethyl)-2-nitro-phenoxy]tetrahydropyran-2-yl]methyl acetate

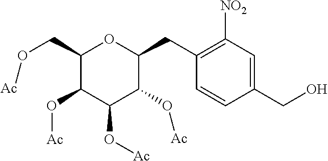 [SMILES: CC(=O)OC[C@H]1O[C@@H](Cc2ccc(CO)cc2[N+](=O)[O-])[C@H](OC(C)=O)[C@@H](OC(C)=O)[C@H]1OC(C)=O]

The title compound was prepared according to Ghosh Ajit, K., at al. A daunorubicin b-galactoside prodrug for use in conjunction with gene-directed enzyme prodrug therapy Tetrahedron Lett. 2000, 41, 4871-4874. LC-MS 517.2 [M+H₂O]⁺, RT 3.81 min.

### Example 6—Intermediary [(2R,3S,4S,5R,6S)-3,4,5-triacetoxy-6-[4-(chlorocarbonyloxymethyl)-2-nitro-phenoxy]tetrahydropyran-2-yl]methyl acetate

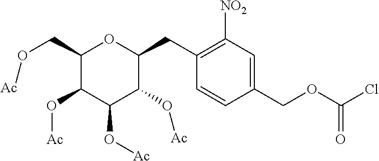 [SMILES: CC(=O)OC[C@H]1O[C@@H](Cc2ccc(COC(=O)Cl)cc2[N+](=O)[O-])[C@H](OC(C)=O)[C@@H](OC(C)=O)[C@H]1OC(C)=O]

The crude title compound was prepared following the method in Example 3 above, for intermediary [(2R,3S,4S,5R,6S)-3,4,5-triacetoxy-6-[4-(hydroxymethyl)-2-nitro-phenoxy]tetrahydropyran-2-yl]methyl acetate.

Example 7—Compound [I], [(2R,3S,4S,5R,6S)-3,4,5-triacetoxy-6-[4-[[(2S,3R,4S,6R)-4-(dimethylamino)-2-[[(2R,3S,4R,5R,8R,10R,11R,12S,13S, 14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxycarbonyloxymethyl]-2-nitro-phenoxy]tetrahydropyran-2-yl]methyl acetate. As can be seen, the galactose moiety is joined to Compound [I] via the hydroxide on the desosamine ring.

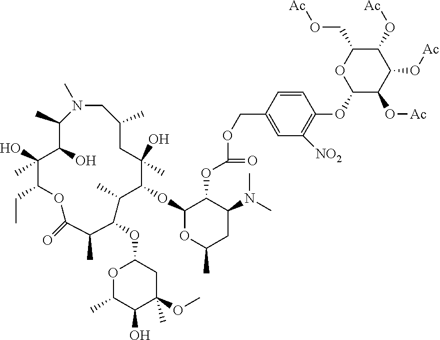 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)CN(C)[C@H](C)[C@@H](O)[C@]1(C)O]

To a stirred ice cold suspension of crude [(2R,3S,4S,5R,6S)-3,4,5-triacetoxy-6-[4-(chlorocarbonyloxymethyl)-2-nitro-phenoxy]tetrahydropyran-2-yl]methyl acetate (0.28 g, 0.50 mmol) and K₂CO₃ (0.14 g, 1.00 mmol) in dry MeCN (6 ml) under nitrogen atmosphere a solution of (2R,3S,4R,5R,8R,10R,11R,12S,13S,14R)-11-[(2S,3R,4S,6R)-4-(dimethylamino)-3-hydroxy-6-methyl-tetrahydropyran-2-yl]oxy-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-1-oxa-6-azacyclopentadecan-15-one (0.23 g, 0.3 mmol) in dry MeCN (4 ml). The reaction mixture was stirred at room temperature for 16 hours. The solid residue was removed by filtration and the solvent was evaporated under reduced pressure to yield a crude product. Purification on silica gel (2-10% MeOH in DCM) afforded [(2R,3S,4S,5R,6S)-3,4,5-triacetoxy-6-[4-[[(2S,3R,4S,6R)-4-(dimethylamino)-2-[[(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxycarbonyloxymethyl]-2-nitro-phenoxy]tetrahydropyran-2-yl]methyl acetate (0.090 g). LC-MS 638.1 [M/2+1]⁺, 1274.7 [M]⁺, RT 4.20 min.

### Example 8—(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-11-[(2S,3R,4S,6R)-3-hydroxy-6-methyl-4-(methylamino)tetrahydropyran-2-yl]oxy-3,5,6,8,10,12,14-heptamethyl-1-oxa-6-azacyclopentadecan-15-one

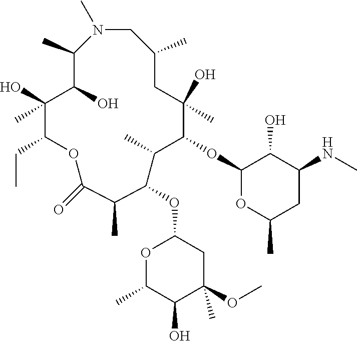 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](NC)[C@H]2O)[C@](C)(O)C[C@@H](C)CN(C)[C@H](C)[C@@H](O)[C@]1(C)O]

The title compound was prepared according to Vujasinovic, Ines et al. Novel tandem Reaction for the Synthesis of N′-Substituted 2-Imino-1,3-oxazolidines from Vicinal (sec-or tert-)Amino Alcohol of Desosamine. Eur. J. Org. Chem. 2011, 2507-2518. LC-MS 735.3 [M+H]⁺, RT 0.97 min.

### Example 9—Compound [IV], has an IUPAC name of [(2R,3S,4S,5R,6S)-3,4,5-triacetoxy-6-[4-[[[(2S,3R,4S,6R)-2-[[(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-3-hydroxy-6-methyl-tetrahydropyran-4-yl]-methyl-carbamoyl]oxymethyl]-2-nitro-phenoxy]tetrahydropyran-2-yl]methyl acetate

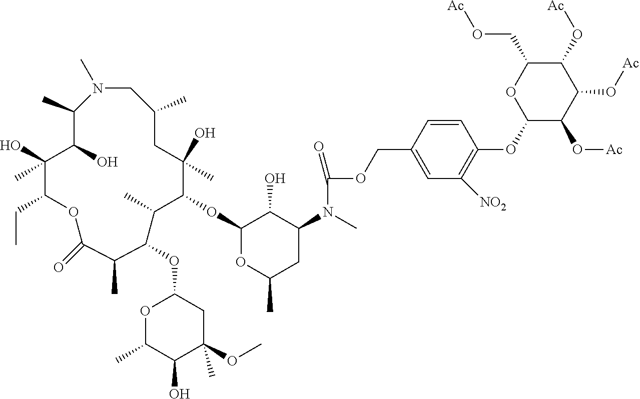 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C(=O)OCc3ccc(O[C@@H]4O[C@H](COC(C)=O)[C@H](OC(C)=O)[C@H](OC(C)=O)[C@H]4OC(C)=O)c([N+](=O)[O-])c3)[C@H]2O)[C@](C)(O)C[C@@H](C)CN(C)[C@H](C)[C@@H](O)[C@]1(C)O]

The title compound (0.064 g) was prepared following the method in Example 7 above, for (2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-11-[(2S,3R,4S,6R)-3-hydroxy-6-methyl-4-(methylamino)tetrahydropyran-2-yl]oxy-3,5,6,8,10,12,14-heptamethyl-1-oxa-6-azacyclopentadecan-15-one and [(2R,3S,4S,5R,6S)-3,4,5-triacetoxy-6-[4-(chlorocarbonyloxymethyl)-2-nitro-phenoxy]tetrahydropyran-2-yl]methyl acetate. LC-MS 1260.5 [M]⁺, RT 5.06 min.

### Example 11—4-[(2S,3R,4S,6R)-4-(dimethylamino)-2-[[(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxy-4-oxo-butanoic acid

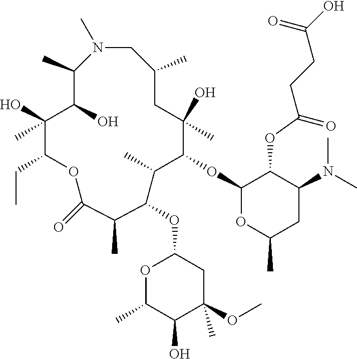 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)CCC(=O)O)[C@](C)(O)C[C@@H](C)CN(C)[C@H](C)[C@@H](O)[C@]1(C)O]

To a stirred solution of (2R,3S,4R,5R,8R,10R,11R,12S,13S, 14R)-11-[(2S,3R,4S,6R)-4-(dimethylamino)-3-hydroxy-6-methyl-tetrahydropyran-2-yl]oxy-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-1-oxa-6-azacyclopentadecan-15-one (0.795 g, 1.00 mmol) in dry DCM (3 ml), DMAP (0.024 g. 0.2 mmol) and succinic anhydride (0.20 g, 2.00 mmol) was added. The reaction mixture was stirred at room temperature under nitrogen atmosphere for 20 hours. The solvent was evaporated under reduced pressure to yield 4-[(2S,3R,4S,6R)-4-(dimethylamino)-2-[[(2R,3S,4R,5R,8R,10R,11R,12S,13S, 14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxy-4-oxo-butanoic acid as a white solid (0.84 g). LC-MS 425.5 [M/2+1]⁺, 849.6 [M]⁺, RT 2.40 min.

### Example 12—O4-[(2S,3R,4S,6R)-4-(dimethylamino)-2-[[(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl] O1-[[3-nitro-4-[(2S,3R,4S,5S,6R)-3,4,5-triacetoxy-6-(acetoxymethyl)tetrahydropyran-2-yl]oxy-phenyl]methyl]butanedioate

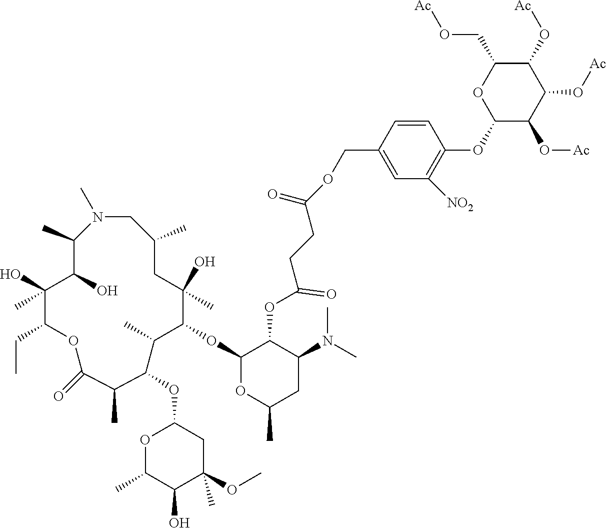 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)CCC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)CN(C)[C@H](C)[C@@H](O)[C@]1(C)O]

To a stirred suspension of 4-[(2S,3R,4S,6R)-4-(dimethylamino)-2-[[(2R,3S,4R,5R,8R,10R,11R,12S,13S, 14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxy-4-oxo-butanoic acid (0.30 g, 0.35 mmol) and [(2R,3S,4S,5R,6S)-3,4,5-triacetoxy-6-[4-(hydroxymethyl)-2-nitro-phenoxy]tetrahydropyran-2-yl]methyl acetate (0.175 g, 0.35 mmol) in dry DCM (8 ml) at room temperature and under nitrogen atmosphere EDC HCl (0.10 g, 0.52 mmol), TEA (50 μl, 0.35 mmol) was added. The reaction mixture was stirred at room temperature under nitrogen atmosphere for 72 hours. The solvent was evaporated under reduced pressure to yield a crude product. Purification on silica gel (5-10% IPA in DCM) afforded O4-[(2S,3R,4S,6R)-4-(dimethylamino)-2-[[(2R,3S,4R,5R,8R,10R,11R,12S,13S, 14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl] O1-[[3-nitro-4-[(2S,3R,4S,5S,6R)-3,4,5-triacetoxy-6-(acetoxymethyl)tetrahydropyran-2-yl]oxy-phenyl]methyl]butanedioate. (0.239 g). LC-MS 660.3 [M/2+1]⁺, 1330.9 [M]⁺, RT 3.33 min.

### Example 13—Compound [VI], 2R,3S,4S,5R,6S)-3,4,5-triacetoxy-6-[4-[[(2S,3R,4S,6R)-4-(dimethylamino)-2-[[(3R,4S,5S,6R,7R,9R,11R,12R,13S,14R)-14-ethyl-7,12,13-trihydroxy-4-[(2S,4R,5S,6S)-5-hydroxy-4-methoxy-4,6-dimethyl-tetrahydropyran-2-yl]oxy-3,5,7,9,11,13-hexamethyl-2,10-dioxo-oxacyclotetradec-6-yl]oxy]-6-methyl-tetrahydropyran-3-yl]oxycarbonyloxymethyl]-2-nitro-phenoxy]tetrahydropyran-2-yl]methyl acetate

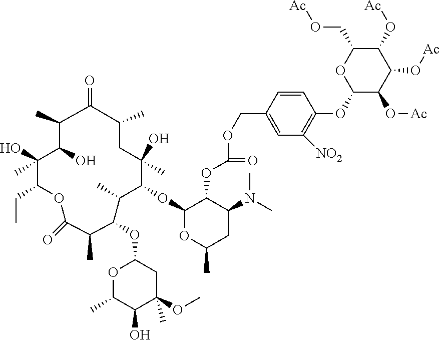 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)C(=O)[C@H](C)[C@@H](O)[C@]1(C)O]

The title compound (0.107 g) was prepared following the method in Example 7 from (3R,4S,5S,6R,7R,9R,11R,12R,13S,14R)-6-[(2S,3R,4S,6R)-4-(dimethylamino)-3-hydroxy-6-methyl-tetrahydropyran-2-yl]oxy-14-ethyl-7,12,13-trihydroxy-4-[(2S,4R,5S,6S)-5-hydroxy-4-methoxy-4,6-dimethyl-tetrahydropyran-2-yl]oxy-3,5,7,9,11,13-hexamethyl-oxacyclotetradecane-2,10-dione and [(2R,3S,4S,5R,6S)-3,4,5-triacetoxy-6-[4-(chlorocarbonyloxymethyl)-2-nitro-phenoxy]tetrahydropyran-2-yl]methyl acetate. LC-MS 1259.8 [M]⁺, RT 4.06 min.

### Example 14—Compound [V], [(2R,3S,4S,5R,6S)-3,4,5-triacetoxy-6-[4-[[(2S,3R,4S,6R)-4-(dimethylamino)-2-[[(3R,4S,5S,6R,7R,9R,10E,11S,12R,14R)-14-ethyl-7,12,13,13-tetrahydroxy-4-[(2S,4R,5S,6S)-5-hydroxy-4-methoxy-4,6-dimethyl-tetrahydropyran-2-yl]oxy-10-(2-methoxyethoxymethoxyimino)-3,5,7,9,11-pentamethyl-2-oxo-oxacyclotetradec-6-yl]oxy]-6-methyl-tetrahydropyran-3-yl]oxycarbonyloxymethyl]-2-nitro-phenoxy]tetrahydropyran-2-yl]methyl acetate

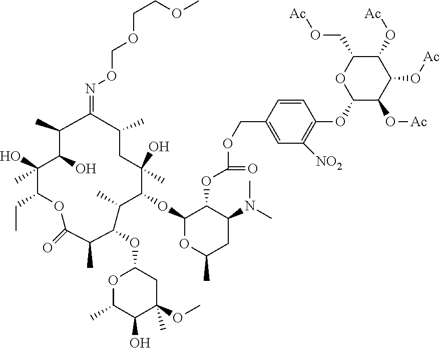 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)/C(=N\OCOCCOC)[C@H](C)[C@@H](O)[C@]1(C)O]

The title compound (0.123 g) was prepared following the method in Example 7 from (3R,4S,5S,6R,7R,9R,10E,11S,12R,14R)-6-[(2S,3R,4S,6R)-4-(dimethylamino)-3-hydroxy-6-methyl-tetrahydropyran-2-yl]oxy-14-ethyl-7,12,13,13-tetrahydroxy-4-[(2S,4R,5S,6S)-5-hydroxy-4-methoxy-4,6-dimethyl-tetrahydropyran-2-yl]oxy-10-(2-methoxyethoxymethoxyimino)-3,5,7,9,11-pentamethyl-oxacyclotetradecan-2-one and [(2R,3S,4S,5R,6S)-3,4,5-triacetoxy-6-[4-(chlorocarbonyloxymethyl)-2-nitro-phenoxy]tetrahydropyran-2-yl]methyl acetate expect IPA was used instead of MeOH as chromatography eluent. LC-MS 682.3 [M/2+1]⁺, 1363.0 [M]⁺, RT 4.32 min.

### Example 15—[(2R,3R,4S,5R,6S)-3,4,5-triacetoxy-6-[4-[[(2S,3R,4S,6R)-4-(dimethylamino)-2-[[(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxycarbonyloxymethyl]-2-nitro-phenoxy]tetrahydropyran-2-yl]methyl acetate

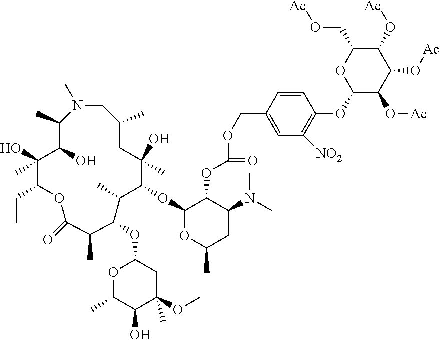 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](COC(C)=O)[C@H](OC(C)=O)[C@H](OC(C)=O)[C@H]3OC(C)=O)c([N+](=O)[O-])c2)[C@](C)(O)C[C@@H](C)CN(C)[C@H](C)[C@@H](O)[C@]1(C)O]

The title compound (0.046 g) was prepared following the method in Example 7 from [(2R,3R,4S,5R,6S)-3,4,5-triacetoxy-6-[4-(chlorocarbonyloxymethyl)-2-nitro-phenoxy]tetrahydropyran-2-yl]methyl acetate and (2R,3S,4R,5R,8R,10R,11R,12S,13S,14R)-11-[(2S,3R,4S,6R)-4-(dimethylamino)-3-hydroxy-6-methyl-tetrahydropyran-2-yl]oxy-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-1-oxa-6-azacyclopentadecan-15-one expect IPA was used instead of MeOH as chromatography eluent. LC-MS 638.3 [M/2+1]⁺, 1274.9 [M]⁺, RT 3.30 min.

### Example 16 [(2S,3R,4S,6R)-4-(dimethylamino)-2-[[(2R,3S,4R,5R,8R,10R,11R,12S,13S,14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl][3-nitro-4-[(2S,3R,4S,5R,6R)-3,4,5-trihydroxy-6-(hydroxymethyl)tetrahydropyran-2-yl]oxy-phenyl]methyl carbonate

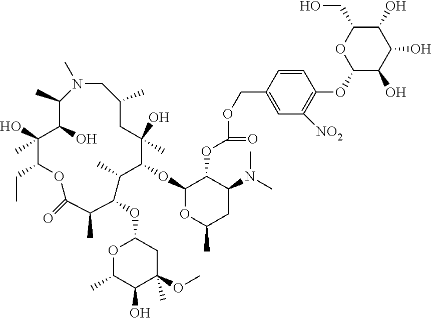 [SMILES: CC[C@H]1OC(=O)[C@H](C)[C@@H](O[C@@H]2C[C@@](C)(OC)[C@@H](O)[C@H](C)O2)[C@H](C)[C@@H](O[C@@H]2O[C@H](C)C[C@H](N(C)C)[C@H]2OC(=O)OCc2ccc(O[C@@H]3O[C@H](CO)[C@H](O)[C@H](O)[C@H]3O)c([N+](=O)[O-])c2)[C@](C)(O)C[C@@H](C)CN(C)[C@H](C)[C@@H](O)[C@]1(C)O]

To a stirred ice cold suspension of [(2R,3S,4S,5R,6S)-3,4,5-triacetoxy-6-[4-[[(2S,3R,4S,6R)-4-(dimethylamino)-2-[[(2R,3S,4R,5R,8R,10R,11R,12S,13S, 14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl]oxycarbonyloxymethyl]-2-nitro-phenoxy]tetrahydropyran-2-yl]methyl acetate (0.050 g, 0.04 mmol) in dry THF (1 ml) under nitrogen atmosphere a solution 0.5M sodium methoxide solution (0.4 ml, 0.20 mmol) was added and the mixture was stirred at 0° C. for 2.5 hours. The reaction mixture was treated with DOWEX 50WX2 (100 mg) at 0° C. for 0.5 hours, the solid resin was removed by filtration, washed with DCM (10 ml) and the solvent was evaporated under reduced pressure to yield [(2S,3R,4S,6R)-4-(dimethylamino)-2-[[(2R,3S,4R,5R,8R,10R,11R,12S,13S, 14R)-2-ethyl-3,4,10-trihydroxy-13-[(2S,4R,5S,6S)-5-hydroxy-4-methoxy-4,6-dimethyl-tetrahydropyran-2-yl]oxy-3,5,6,8,10,12,14-heptamethyl-15-oxo-1-oxa-6-azacyclopentadec-11-yl]oxy]-6-methyl-tetrahydropyran-3-yl][3-nitro-4-[(2S,3R,4S,5R,6R)-3,4,5-trihydroxy-6-(hydroxymethyl)tetrahydropyran-2-yl]oxy-phenyl]methyl carbonate (0.050 g). LC-MS 554.0 [M/2+1]⁺, RT 3.52 min.

The therapeutic agents may be used in the form of pharmaceutical compositions which may be prepared using one or more known methods. For example, a pharmaceutical composition may be prepared by using diluents or excipients such as, for example, one or more fillers, bulking agents, binders, wetting agents, disintegrating agents, surface active agents, lubricants, and the like as are known in the art. Various types of administration unit forms can be selected depending on the therapeutic purpose(s). Examples of forms for pharmaceutical compositions include, but are not limited to, tablets, pills, powders, liquids, suspensions, emulsions, granules, capsules, suppositories, injection preparations (solutions and suspensions), topical creams, and other forms as may be known in the art. For the purpose of shaping a pharmaceutical composition in the form of tablets, any excipients which are known may be used, for example carriers such as lactose, white sugar, sodium chloride, glucose, urea, starch, calcium carbonate, kaolin, cyclodextrins, crystalline cellulose, silicic acid and the like; binders such as water, ethanol, propanol, simple syrup, glucose solutions, starch solutions, gelatin solutions, carboxymethyl cellulose, shelac, methyl cellulose, potassium phosphate, polyvinylpyrrolidone, etc. Additionally, disintegrating agents such as dried starch, sodium alginate, agar powder, laminalia powder, sodium hydrogen carbonate, calcium carbonate, fatty acid esters of polyoxyethylene sorbitan, sodium laurylsulfate, monoglyceride of stearic acid, starch, lactose, etc., may be used. Disintegration inhibitors such as white sugar, stearin, coconut butter, hydrogenated oils; absorption accelerators such as quaternary ammonium base, sodium laurylsulfate, etc., may be used. Wetting agents such as glycerin, starch, and others known in the art may be used. Adsorbing agents such as, for example, starch, lactose, kaolin, bentonite, colloidal silicic acid, etc., may be used. Lubricants such as purified talc, stearates, boric acid powder, polyethylene glycol, etc., may be used. If tablets are desired, they can be further coated with the usual coating materials to make the tablets as sugar coated tablets, gelatin film coated tablets, tablets coated with enteric coatings, tablets coated with films, double layered tablets, and multi-layered tablets. Pharmaceutical compositions adapted for topical administration may be formulated as ointments, creams, suspensions, lotions, powders, solutions, pastes, gels, foams, sprays, aerosols, or oils. Such pharmaceutical compositions may include conventional additives which include, but are not limited to, preservatives, solvents to assist drug penetration, co-solvents, emollients, propellants, viscosity modifying agents (gelling agents), surfactants, and carriers.

The present approach may be used to prevent and/or reduce the likelihood of tumor recurrence, metastasis. Anti-cancer treatments often fail because the tumor recurs or metastasizes, particularly after surgery. CSC mitochondrial activity is, at least in part, responsible for these causes of treatment failure. Embodiments of the present approach may be used in situations where conventional cancer therapies fail, and/or in conjunction with anti-cancer treatments to prevent or reduce the likelihood of failure due to tumor recurrence and/or metastasis.

The present approach provides for methods of selectively targeting cancer cells. The target cancer cell may be at least one of a CSC, an energetic cancer stem cell (e-CSC), a circulating tumor cell (CTC, a seed cell leading to the subsequent growth of additional tumors in distant organs, a mechanism responsible for a large fraction of cancer-related deaths), and a therapy-resistant cancer cell (TRCC, a cell that has developed a resistance to one or more of chemotherapies, radiotherapies, and other common cancer treatments).

As described in Applicant's co-pending U.S. Provisional Patent Application Nos. 62/686,881, filed Jun. 19, 2018, and 62/731,561, filed Sep. 14, 2018, and incorporated by reference in their entirety, e-CSCs represent a CSC phenotype associated with proliferation. In addition to bulk cancer cells and CSCs, it should be appreciated that the present approach may be used to target a hyper-proliferative cell sub-population that the inventors refer to as e-CSCs, which show progressive increases in stemness markers (ALDH activity and mammosphere-forming activity), highly elevated mitochondrial mass, and increased glycolytic and mitochondrial activity.

In view of the foregoing, it should be appreciated that the present approach may take a wide variety of forms, depending on the embodiment. For example, embodiments of the present approach may take the form of a composition, and in particular a pharmaceutical composition. A compound of the present approach may be the active ingredient in the composition, and may be present in a pharmaceutically-effective amount along with one or more pharmaceutically-acceptable excipients. Embodiments of the present approach may also take the form of methods for preventing or reducing the likelihood of at least one of tumor recurrence and metastasis. In some embodiments, a pharmaceutically-effective amount of a composition having, as a therapeutic agent, a compound of the present approach and one or more pharmaceutically-acceptable excipients, may be administered. In some embodiments, an effective amount of a composition having, as its therapeutic agent, an embodiment of a compound as described herein may be administered.

The following paragraphs describe the materials and assays used to generate the data described herein. It should be appreciated that the person having an ordinary level of skill in the art may perform the same assays as described herein, and/or may utilize other assays generally known in the art to assess the physical, chemical, and pharmaceutical properties of a compound as described herein.

Reagents and Model Cell Lines: It should be apparent that other cell lines may be used without departing from the present approach. MRC-5 (ATCC® CCL-171) human lung fibroblast cells, BJ (ATCC® CRL2522) human skin fibroblasts, and MCF-7 human breast adenocarcinoma cells were purchased from the ATCC (American Type Culture Collection). hTERT-BJI cells were from Clontech, Inc. MCF-7 and hTERT-BJ 1 cells were grown in DMEM supplemented with 10% fetal bovine serum, GlutaMAX and 1% penicillin-streptomycin and incubated at 37 C in a humidified 5% CO2 incubator. The medium was changed 2-3 times/week. Gibco-brand cell culture media (MEM) was purchased from Life Technologies.

Bromodeoxyuridine, azithromycin, roxithromycin and erythromycin were purchased from Sigma-Aldrich.

BrdU Assay: Cells were plated into 24-well plates. Next day, half of the plate was treated with 100 μM of BrdU while control wells were treated with vehicle only (DMSO) and incubated for 8 days at 37° C. in a 5% CO2 humidified atmosphere. After 8 days of BrdU treatment cells were treated with various test compounds or drugs (e.g., azithromycin, roxithromycin, telithromycin, erythromycin, etc.) for another 3-5 days. BrdU or vehicle treatments were continued during the drug treatments as well.

Sulphorhodamine B assay: After the incubation of the plates cell viability was measured by Sulphorhodamine B assay (SRB). The assay is based on the measurement of cellular protein contents. Cells were fixed with 10% Trichloroacetic acid (TCA) for 1 hour at 40 C, and were dried overnight at room temperature. Then, plates were incubated with SRB for 30 min, washed twice with 1% acetic acid and air dried for at least 1 h. Finally, the protein-bound dye was dissolved in a 10 mM Tris, pH 8.8, solution and read using a plate reader at 540-nm.

xCELLigence Assay System: xCELLigence RTCA System (ACEA Biosciences Inc.). Briefly, MRC-5 lung fibroblasts (vehicle alone and/or treated with 100 μM BrdU) were seeded in each well and employed to assess the efficacy of Azithromycin, using RTCA (real-time cell analysis), via the measurement of cell-induced electrical impedance. This approach allows the quantification of the onset and kinetics of the cellular response. Experiments were repeated several times independently, using quadruplicate samples for each condition.

Autophagy and Cell cycle analysis: Autophagy (using Muse™ Autophagy LC3-antibody based Kit, Merck Millipore) and cell cycle (Muse® Cell Cycle Kit, Merck Millipore) experiments were performed according to manufacturer's instructions.

Beta-Gal staining: Beta-Galactosidase staining of BrdU-treated MRC-5 cells was performed by Senescence β-Galactosidase Staining Kit (#9860, Cell Signalling Technology Inc.) and was done according to manufacturer's protocol.

3D Anchorage Independent Growth Assay: This assay is also referred to as the mammosphere formation assay. A single-cell suspension was prepared using enzymatic, and manual disaggregation (25-g needle). Then, cells were plated at a density of 500 cells/cm² in mammosphere medium (DMEM-F12+1X B-27 Plus Supplement+20 ng/ml EGF+Pen/Strep) under non-adherent conditions, in culture dishes pre-coated with (2-hydroxyethylmethacrylate) (poly-HEMA, Sigma Aldrich Inc.), called “mammosphere plates.” Cells were grown for 5 days and maintained in a humidified incubator at 37° C. After 5 days of culture, 3D-mammospheres >50 μm were counted using an eye piece (“graticule”), and the percentage of cells plated which formed spheres was calculated and is referred to as percent mammosphere formation, and was normalized to one (1=100% MFE). 3D mammosphere formation efficiency (MFE) was analyzed in both the ATP-low and ATP-high sub-populations of cells. All 3D mammosphere experiments were performed in triplicate, at least 3 times independently.

Statistical Analysis: All analyses were performed with GraphPad Prism 6. Data were represented as mean±SD (or ±SEM where indicated). All experiments were conducted at least 3 times independently, with >3 technical replicates for each experimental condition tested (unless stated otherwise, e.g., when representative data is shown). Statistically significant differences were determined using the Student's t-test or the analysis of variance (ANOVA) test. For the comparison among multiple groups, one-way ANOVA was used to determine statistical significance. p<0.05 was considered significant.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The invention includes numerous alternatives, modifications, and equivalents as will become apparent from consideration of the following detailed description.

It will be understood that although the terms “first,” “second,” “third,” “a),” “b),” and “c),” etc. may be used herein to describe various elements of the invention, and the claims should not be limited by these terms. These terms are only used to distinguish one element of the invention from another. Thus, a first element discussed below could be termed an element aspect, and similarly, a third without departing from the teachings of the present invention. Thus, the terms “first,” “second,” “third,” “a),” “b),” and “c),” etc. are not intended to necessarily convey a sequence or other hierarchy to the associated elements but are used for identification purposes only. The sequence of operations (or steps) is not limited to the order presented in the claims.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also, as used herein, “and/or” refers to and encompasses any and all possible combinations of one or more of the associated listed items, as well as the lack of combinations when interpreted in the alternative (“or”).

The terms “decrease,” “lower,” “lessen,” and “reduce” generally refer to the ability of compositions according to the present approach to produce and/or cause a lesser physiological response (i.e., a measurable downstream effect), as compared to the response caused by either vehicle or a control molecule/composition, e.g., decreased tumor volume. A “decrease” or “reduced” response is typically a “statistically significant” response, and may include an decrease that is 1.1, 1.2, 1.5, 2, 3, 4, 5, 6, 7, 8, 9, 10, 15, 20, 30, or more times (e.g., 500, 1000 times) (including all integers and decimal points in between and above 1, e.g., 1.5, 1.6, 1.7. 1.8, etc.) the response produced by normal, untreated, or control-treated subject.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

The term “about,” as used herein when referring to a measurable value, such as, for example, an amount or concentration and the like, is meant to encompass variations of ±20%, ±10%, ±5%, ±1%, ±0.5%, or even ±0.1% of the specified amount. A range provided herein for a measurable value may include any other range and/or individual value therein.

Having thus described certain embodiments of the present invention, it is to be understood that the invention defined by the appended claims is not to be limited by particular details set forth in the above description as many apparent variations thereof are possible without departing from the spirit or scope thereof as hereinafter claimed.

What is claimed is:

1. A compound having the chemical structure of one of Compound [I] and Compound [II]: 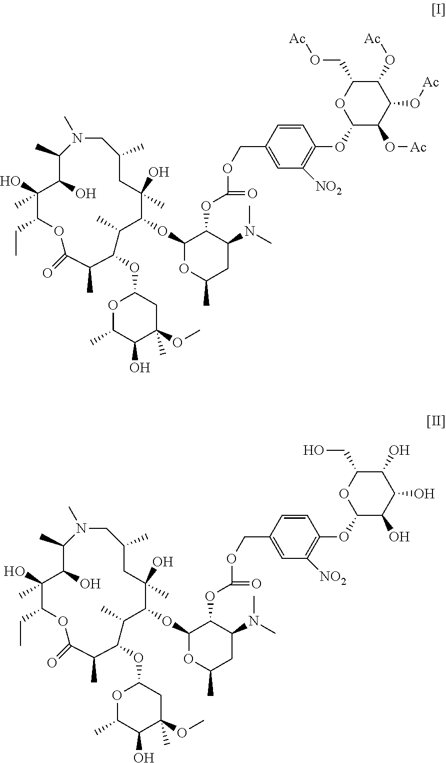
or a pharmaceutically acceptable salt thereof.

2. A pharmaceutical composition comprising a pharmaceutically effective amount of a compound having the chemical structure of one of Compound [I] and Compound [II], or a pharmaceutically acceptable salt thereof, and a pharmaceutically acceptable carrier.

3. A method for treating or preventing tumor recurrence and/or metastasis, the method comprising administering to a patient at risk of tumor recurrence and/or metastasis a pharmaceutically effective amount of the pharmaceutical composition of claim 2.

4. The method of claim 3, wherein the administering is performed at least one of prior to a cancer treatment, with a cancer treatment, and following a cancer treatment.

5. A method for inhibiting the propagation of cancer stem cells in a patient, the method comprising administering to the patient a pharmaceutically effective amount of the pharmaceutical composition of claim 2.

6. The method of claim 5, wherein the administering is performed at least one of prior to a cancer treatment, with a cancer treatment, and following a cancer treatment.

7. A method for treating cancer, the method comprising: administering to a person having cancer a pharmaceutically effective amount of the pharmaceutical composition of claim 2.

8. The method of claim 7, wherein the administering is performed at least one of prior to a cancer treatment, with a cancer treatment, and following a cancer treatment.

9. A method for eradicating senescent cells, the method comprising administering to a patient a pharmaceutically effective amount of the pharmaceutical composition of claim 2.

10. A method for minimizing the accumulation of senescent cells in a subject, the method comprising administering to the subject a pharmaceutically effective amount of the pharmaceutical composition of claim 2.

11. A method for delaying the onset of senescence in a subject, the method comprising administering to the subject a pharmaceutically effective amount of the pharmaceutical composition of claim 2.

12. A method for treating the effects of aging in a subject, the method comprising administering to the subject a pharmaceutically effective amount of the pharmaceutical composition of claim 2.

13. An anti-aging therapeutic method comprising administering to a subject a pharmaceutically effective amount of the pharmaceutical composition of claim 2.
